SEQUENCE LISTING (7 sequences)

SEQ ID NO: 1 - DNA, 2713 bp, Mus musculus
  5′UTR (1)..(72)
  CDS (73)..(1272)
  intron (163)..(164)
  3′UTR (1273)..(2713)
  polyA_signal (2219)..(2224)
  polyA_signal (2693)..(2698)
ccctgtgctcagagtagggctccgagtttcgaaccactggtggcggattgcccgcccgccccacgtccggggatgcgaagtctcagcctggcgtggctgctgggaggtatcacccttctggcggcctcggtctcctgcagccggaccgagaaccttgcaccgggacgcaacaacagtaaaggaagaagtcttattggcagattagaaacccagcctccaatcactgggaaaggggttccggtagaaccaggcttttccatcgatgagttctctgcgtccatcctcaccgggaagctgaccacggtctttcttccggtcgtctacattattgtgtttgtgattggtttgcccagtaatggcatggccctctggatcttccttttccgaacgaagaagaaacaccccgccgtgatttacatggccaacctggccttggccgacctcctctctgtcatctggttccccctgaagatctcctaccacctacatggcaacaactgggtctacggggaggccctgtgcaaggtgctcattggctttttctatggtaacatgtattgctccatcctcttcatgacctgcctcagcgtgcagaggtactgggtgatcgtgaaccccatgggacaccccaggaagaaggcaaacatcgccgttggcgtctccttggcaatctggctcctgatttttctggtcaccatccctttgtatgtcatgaagcagaccatctacattccagcattgaacatcaccacctgtcacgatgtgctgcctgaggaggtattggtgggggacatgttcaattacttcctctcactggccattggagtcttcctgttcccggccctccttactgcatctgcctacgtgctcatgatcaagacgctccgctcttctgctatggatgaacactcagagaagaaaaggcagagggctatccgactcatcatcaccgtgctggccatgtacttcatctgctttgctcctagcaaccttctgctcgtagtgcattatttcctaatcaaaacccagaggcagagccacgtctacgccctctaccttgtcgccctctgcctgtcgaccctcaacagctgcatagacccctttgtctattactttgtctcaaaagatttcagggatcacgccagaaacgcgctcctctgccgaagtgtccgcactgtgaatcgcatgcaaatctcgctcagctccaacaagttctccaggaagtccggctcctactcttcaagctcaaccagtgttaaaacctcctactgagctgtacctgaggatgtcaagcctgcttgatgatgatgatgatgatggtgtgtgtgtgtgtgtgtgtgtgtgtgtgtgtgtgtgtgtgcacccgtgtgtgagtgcgtggtagggatgcaccaacatgcatggggctgtcatttcctatccaagctgtctgtctctgcaccaatcacaagcatgcagctctccccaggattgccagaagcctcctcctttgcatgagaacagtcttccactctgatgaaaagcatcagtatcagaaactgaaacgaactgagaggagcttgttttgtgaaagtgaagagaagatggagggtcagtgacttgcaaaaaaaaccaaccaaacaaaaactacacctggcaagaaggctaagactctctgaaatgcttccttttccatctggagttcgtctcggccttgttcaggacctgaggccctggtagagcttcagtccagttgattgactttacagacttgagagaggagtgaatgaggagtgaatgaggctcctggcggcatcctaaccggctaacagtggccttgctggacaataggattcagatggctggagttacattctcacaccatttcatcagaactattggggatcttgatcaatgtgcaggtcccttagcgtcagtaaccctgggagctcagacacgatgggggtgagggtgggggtgggggtgggggtgaggctctacaaaccttagtgatgactgcagacacagaaccatggagctgagcctgcttctgcttgccagggcaccactgtaatgttggcaaagaaaaaccaacagcagtgttttgagcctctttttttggtcagtttatgatgaatttgcctattggtttattgggattttcagttcctttattactttgttgtaattttgtgtgtttattagtcaagaaaaagaagatgaggctcttaaaaatgtaaataaaatttttggttttttggttttttaacttgggccaactacaaatactgcttaggtttttttctaacttaattgttaactacatcatgtgaacttaagacattttcatgataaagcattactgtagtgtcagttttccctcatcctcgatcatagtccttcccgtgaagcagggcccttcccctcccccccctttgccgtttccctccccaccagatagtcccctgtctgctttaacctaccagttagtattttataaaaacagatcattggaatatttattatcagttttgttcactgttatcagttttgttcactaatttgtccaataatggaattaacgtcttctcatctgtttgaggaagatctgaaacaaggggccattgcaggagtacatggctccaggcttactttatatactgcctgtatttgtggctttaaaaaaatgaccttgttatatgaatgctttataaataaataatgcatgaacttt

SEQ ID NO: 2 - protein, 399 aa, Mus musculus
MRSLSLAWLLGGITLLAASVSCSRTENLAPGRNNSKGRSLIGRLETQPPITGKGVPVEPGFSIDEFSASILTGKLTTVFLPVVYIIVFVIGLPSNGMALWIFLFRTKKKHPAVIYMANLALADLLSVIWFPLKISYHLHGNNWVYGEALCKVLIGFFYGNMYCSILFMTCLSVQRYWVIVNPMGHPRKKANIAVGVSLAIWLLIFLVTIPLYVMKQTIYIPALNITTCHDVLPEEVLVGDMFNYFLSLAIGVFLFPALLTASAYVLMIKTLRSSAMDEHSEKKRQRAIRLIITVLAMYFICFAPSNLLLVVHYFLIKTQRQSHVYALYLVALCLSTLNSCIDPFVYYFVSKDFRDHARNALLCRSVRTVNRMQISLSSNKFSRKSGSYSSSSTSVKTSY

SEQ ID NO: 3 - protein, 6 aa, Homo sapiens
  C-term amidated
SFLLRN

SEQ ID NO: 4 - protein, 6 aa, Homo sapiens
  C-term amidated
SLIGKV

SEQ ID NO: 5 - protein, 6 aa, Mus musculus
  C-term amidated
SLIGRL

SEQ ID NO: 6 - protein, 6 aa, Homo sapiens
GYPGQV

SEQ ID NO: 7 - protein, 6 aa, Mus musculus
GYPGKF

(19) United States
(12) Patent Application Publication
(10) Pub. No.: US 2006/0183664 A1
(43) Pub. Date: Aug. 17, 2006

(54) Pharmaceutical composition for delayed hypersensitivity

(76) Inventors: Toshiaki Takizawa, Tokyo (JP); Masaki Tamiya, Tokyo (JP); Naohiro Saito, Tokyo (JP); Toru Kanke, Tokyo (JP); Jiro Matsumoto, Saitama (JP); Yasushi Wada, Tokyo (JP); Junichi Kawagoe, Saitama (JP); William R Ferrell, Glasgow (GB); John C Lockhart, Glasgow (GB)

Correspondence Address: EDWARDS & ANGELL, LLP, P.O. BOX 55874, BOSTON, MA, 02205, US

(21) Appl. No.: 10/496,851

(22) PCT Filed: Dec. 13, 2002
(86) PCT No.: PCT/GB02/05658

§ 371 Date: May 27, 2004

(30) Foreign Application Priority Data
Dec. 13, 2001 (GB) 0129848.8
Dec. 31, 2001 (GB) 0131103.4

(51) Int. Cl. A61K 38/54

(52) U.S. Cl. 514/2

(57) ABSTRACT

The present invention provides a pharmaceutical composition by a novel action mechanism without serious side effects for delayed hypersensitivity and a screening method of the same. The invention also provides a novel assay method of inhibitors/suppressive agents of PAR-2. The invention relates to a pharmaceutical composition for delayed hypersensitivity containing one or two or more active ingredients selected from the group consisting of inhibitors of PAR-2 and suppressive agents of PAR-2 gene expression and a pharmaceutically acceptable carrier, and to a method for screening active ingredients for pharmaceutical composition for delayed hypersensitivity by contacting a subject substance with cells expressing PAR-2 and by determining expression or activity of PAR-2. The invention also relates to a method for detecting or quantifying actions of the subject substance for PAR-2 using cells expressing PAR-2 in a culture containing inositol.

### FIELD OF THE INVENTION

The present invention relates to a pharmaceutical composition for delayed hypersensitivity of which active ingredient is an inhibitor of PAR (protease-activated receptor)-2 and/or a suppressive agent for expression of PAR-2 gene. The present invention also relates to a method for screening active ingredients for a pharmaceutical composition for delayed hypersensitivity consisting of screening subject materials for inhibition of PAR-2 or suppression of PAR-2 gene expression. Further, the present invention relates to an assay method for PAR-2 based on production of inositol phosphate as an indicator.

### BACKGROUND OF THE INVENTION

Allergy (hypersensitivity) is a state of a living body where immune reaction is induced in excessive or inappropriate manners and, in some cases, tissue is damaged.

Allergic responses (hypersensitivity) are induced in the second contact with an identical antigen, and classified into two types, i.e., an immediate hypersensitivity in which humoral immunity is involved, and a delayed hypersensitivity in which cellular immunity is involved. Further, Coombs and Gell divided them into I to IV types according to the differences of occurrence mechanisms and symptoms. In the type I of hypersensitivity, immunoglobulin E antibody which binds to mast cells in tissues and basophiles in blood reacts with its specific antigen and then degranulation is induced to release chemical mediators such as histamine. The released chemical mediators act against various organs resulting in acute inflammation. In the type II of hypersensitivity, an antibody binds to an antigen on the surface of self or foreign cells, which are phagocytized, activate killer cells and induce cytolysis involving complements. In the type III of hypersensitivity, immune complexes in which complements bind to antigen/antibody conjugations formed in vivo by entered antigens deposit in tissues, which activate complements, and polymorphonuclear leukocytes are congregated around the deposit sites, resulting in local lesions. In the type IV hypersensitivity, i.e., delayed hypersensitivity, chemical mediators having biological activity such as cytokines are liberated and released by direct reaction between an antigen and T cells capable of reacting specifically to the antigen, resulting in assembling cells in a local tissue to induce inflammation.

The delayed hypersensitivity includes tuberculin reaction, rejection in allogenic transplantation, cellular defense reaction of infection, contact dermal hypersensitivity and the like, and these reactions are known to be suppressed most strongly by steroid drugs. Therefore, the steroid drugs are effective for the diseases caused through delayed hypersensitivity, however, the steroid drugs are problematic in that discontinuation and regimen of medication are difficult because a serious side effect, i.e., dependency on steroids occurs when the steroid drugs are used for a long time. Also, in treatment of contact dermatitis, a contact dermal hypersensitivity with the steroid drugs, various side effects occur by alteration of skin conditions such as atrophy of skin, acne, hirsutism and the like, and the skin conditions conversely deteriorate in some cases.

There are a wide variety of chronic diseases by delayed hypersensitivity. They include those by non-infectious substances such as sarcoidosis and Crohn's disease, and those by infectious microbes such as bacteria, protozoa, fungi and the like. The diseases are believed to be caused by chronic antigen stimuli afforded by these microbes. It is believed that tissue disorders are induced by persistent stimuli although infection is often limited locally by activation of macrophages and the like. These infectious diseases include tuberculosis, lepra (Hansen's disease), schistosomiasis and the like.

On the other hand, PZLR (protease-activated receptor)-2 is one type of PARs belonging to 7-transmembrane G-protein-coupled receptor family. Currently, four types of PARs have been cloned, i.e., PAR-1, PAR-2, PAR-3 and PA-4, and belong to a receptor family which mediates actions of serine proteases such as thrombin, trypsin and the like in various cells. All of PAR-1, PAR-3 and PAR-4 have been demonstrated to have functions as receptors which relate to platelet aggregation by thrombin. However, PAR-2 is functionally distinct from the other PARs since it is activated by trypsin and tryptase but not by thrombin although it has a structure and activation mechanisms in common with other PARs.

In these PARs, the specific site at N-terminus of the amino acid sequence of the receptor is cleaved by the action of thrombin or other proteases, and activation of the receptor is caused by binding of the newly-exposed terminus to the binding site of the receptor per se. The summary of the amino acid sequences at the cut end which activate the receptors is represented by the single character code for amino acids.

| | | |
|---|---|---|
| PAR-1 | SFLLRN-NH₂ | (human) |
| PAR-2 | SLIGKV-NH₂ | (human) |
| | SLIGRL-NH₂ | (mouse) |
| PAR-3 | not avalable | |
| PAR-4 | GYPGQV | (human) |
| | GYPGKF | (mouse) |

PAR-1, PAR-2 and PAR-4 can be activated non-enzymatically by exogenously added peptides having the amino acid sequences of the cut ends, but PAR-3 can not be activated by such foreign peptides. In a recent study, it has been proved that murine PAR-3 is not activated by itself and is a cofactor of PAR-4 which can function only in the presence of PAR-4 (Nature, 404:609-613, 2000).

PAR-2 is known to be activated by tissue factors/VIIa factor; Xa factor; acrosin, a type of sperm proteases; trypsin-like serine protease identified from rat brain; trypsin; tryptase; and synthetic peptides having similar sequence to the ligand of PAR-2.

Several reports using trypsin, tryptase, PAR-2 activating peptides and PAR-2 knockout mice have shown physiological and pathological roles of PAR-2 in respect to inflammation and hypersensitivity (Br. J. Pharmacol., 125: 419-422,1988; Br. J. Pharmacol., 127:1083-1090,1999; Eur. J. Pharmacol., 328:89-97, 1997; J. Pharmacol. Exp. Ther., 288:671-678, 1999; and J. Immunol., 165:6504-6510, 1999).

It has been reported that transient inflammatory responses are induced by an agonist of PAR-2 (SLIGRL-NH₂) (Br. J. Pharmacol. 125:419-422, 1998). However, mRNA for PAR-2 was not detected by a RT-PCR method in peritoneal mast cells, and degranulation by the PAR-2 agonist was not observed in mast cells (Jpn. J. Pharmacol., 82:74-77, 2000). Thus, the transient inflammatory response by the PAR-2 agonist (SLIGRL-NH₂) is believed to be highly potential of secondary response via actions for cells/tissues other than mast cells. It has been also reported that nervous inflammation is exerted not depending on mast cells in experiments using high concentrations of the PAR-2 agonist (SLIGRL-NH₂)(Br J. Pharmacol., 127:1083-1090, 1999; and Nat. Med., 6:151-158, 2000).

However, they are indirect evidences of PAR-2 regarding to immediate hypersensitivity, and nervous inflammation or reports of PAR-2 regarding to circulatory organs, and have not suggested roles of PAR-2 in delayed hypersensitivity.

There is a report that the inhibitor of tryptase is effective for treatment of dermatitis in the murine model of delayed hypersensitivity, but an involvement of PAR-2 is not suggested at all in the report (J. Med. Chem., 41:4854-4860, 1998). Tryptase is known to decompose and activate lots of ligands other than PAR-2, such as VIP (vasoactive intestinal peptide), PHM (peptide histidine methionine), CGRP (calcitonin gene related peptide), fibrinogen, gelatinase, fibronectin, IV type of collagen, MMP (matrix metalloprotease), uPA (urinary type plasminogen activator), and kininogenase (Pharmacol. Rev., 53:245-282, 2001), and which mechanism is effective for treatment of dermatitis in the murine model of delayed hypersensitivity has not been described. The present inventors showed that PAR-2 is scarcely involved in the dermatitis model called passive cutaneous anaphylaxis in which tryptase is released from mast cells in the experiments using PAR-2 knockout mice. The relationship between suppression of PAR-2 activation and delayed hypersensitivity has not been disclosed in this report (J. Med. Chem., 41:4854-4860, 1998).

### SUMMARY OF THE INVENTION

The present invention provides a pharmaceutical composition for delayed hypersensitivity via a novel action mechanism without any serious side effects and methods for screening the same. The invention also provides a novel assay method for inhibitors of PAR-2 and/or suppressive agents of PAR-2 expression.

As the results of intensive study, the present inventors have found that PAR-2 is involved in reactions of delayed hypersensitivity, have found that an inhibitor of PAR-2 and/or a suppressive agent of PAR-2 gene expression is a pharmaceutical composition for delayed hypersensitivity with few manifestation of side effects, and have completed the invention based on these findings.

That is, the present invention relates to a pharmaceutical composition for delayed hypersensitivity containing one or two or more active ingredients selected from the group consisting of inhibitors of PAR-2 and/or suppressive agents of PAR-2 gene expression, and a pharmaceutically acceptable carrier.

The invention also relates to a method for screening active ingredients for pharmaceutical composition for delayed hypersensitivity comprising screening subject materials for inhibitory action against PAR-2 or suppressive action against PAR-2 gene expression by contacting the subject materials to cells on which PAR-2 is expressed followed by determining expression or activity of PAR-2.

Further, the invention relates to a method for detection or quantification of PAR-2 activation by incubation of PAR-2 expressing cells with a medium containing inositol followed by detection or quantification of inositol phosphates in the PAR-2 expressing cells incubated with the medium containing a subject material.

PAR-2 was cloned by Nystedt et al. in 1994 (Proc. Natl. Acad. Sci. USA, 91:9208-9212, 1994). The base sequence of PAR-2 and the amino acid sequence of its coding region are shown in the sequence number 1 in the sequence lists.

The production of PAR-2 gene knockout mice (PAR-2^{-/-} mice) has been reported by Damiano in 1999 (J. Pharmacol. Exp. Ther., 288:671-678, 1999).

The present inventors have found that PAR-2 is involved in reactions of delayed hypersensitivity when analyzing functions of PAR-2 using PAR-2^{-/-} mice.

First, the present inventors examined whether skin atrosphy was exerted by deletion of PAR-2 gene by measuring ear pinna thicknesses of wild type and PAR-2^{-/-} mice.

The average thicknesses of ear pinna in non-treated wild type mice and PAR-2^{-/-} mice are 22.7±0.4(×10⁻² mm; the mean±standard error) and 22.0±0.4(×10⁻² mm; the mean±standard error), respectively, and no statistical difference was observed between them. No visible difference between them was observed with the naked eye, indicating that no skin atrophy was caused by the inhibition of PAR-2.

Next, the models for picryl chloride (PC) induced contact dermatitis and oxazolone (Ox) induced contact dermatitis were tested using PAR-2^{-/-} mice, and then significant difference was observed between wild type and PAR-2^{-/-} mice.

FIG. 1A (upper of FIG. 1) shows the results of ear pinna edema in the model for picryl chloride (PC) induced contact dermatitis, and FIG. 1B (lower of FIG. 1) shows results of ear pinna edema in the model for oxazolone (Ox) induced contact dermatitis. Each vertical axis in FIGS. 1A and 1B denotes the difference of ear pinna thickness before and after the induction when 1% PC-olive oil solution (FIG. 1A) or 0.5% Ox-acetone solution (FIG. 1B) was applied on the both sides of ear pinna to induce edema in the sensitized mice and ear pinna thickness was measured after 24 hours. The left is from the wild type mice and the right is from the PAR-2^{-/-} mice in FIGS. 1A and 1B.

No effect of the solvent (olive oil or acetone) application was observed in these experiments.

In the wild type mice, incidences of edema of which peak was at 24 hours after challenge of PC or Ox were observed, and edema and redness of ear pinnas were macroscopically observed. On the contrary in the PAR-2^{-/-} mice, edema exerted by PC challenge was obviously suppressed (FIG. 1A), and edema exerted by Ox was completely suppressed at 24 hour after the challenge when the peak of ear pinna edema was observed in the wild type mice.

Further, histological examination of these edema was carried out.

The thin slice specimens of both ear pinnas of the mice used for PC- or Ox-induced contact dermatitis model experiments were stained with hematoxylin-eosin to histologically observe the effects of PAR-2 deletion on. PC- or Ox-induced contact dermatitis.

FIG. 2 is color photographs instead of drawings which show histopathological pictures at 24 hours after PC or Ox application to the wild type mice (the left side of FIG. 2, A, C and E) and the PAR-2^{-/-} mice (the right side of FIG. 2, B, D and F). The upper panels of FIG. 2 show the cases of solvent (olive oil) treatment, and the left (FIG. 2A) is from the wild type and the right (FIG. 2B) is from the PAR-2^{-/-} mouse. The middle panels of FIG. 2 (FIGS. 2C and 2D) show the cases of PC treatment and the left (FIG. 2C) is from the wild type and the right (FIG. 2D) is from the PAR-2^{-/-} mouse. The lower panels of FIG. 2 (FIGS. 2E and 2F) show the cases of Ox treatment, and the left (FIG. 2E) is from the wild type and the right (FIG. 2F) is from the PAR-2^{-/-} mouse.

Edema and infiltration by inflammatory cells such as neutrophils, macrophages, lymphocytes, eosinophils and the like were observed by application of PC or Ox on ear pinnas of the wild type mice (FIGS. 2C and 2E). These edema and ininfiltration by inflammatory cells such as neutrophils, macrophages, lymphocytes, eosinophils and the like were remarkably suppressed in the PAR-2^{-/-} mice (FIGS. 2D and 2F). In the histological observation, the incidences of edema were found to be obviously suppressed in the PAR-2^{-/-} mice in these experiments.

Further, the tests were conducted in the model for passive cutaneous anaphylaxis (PCA). Antiserium against ovalbumin (OA) was subcutaneously administered in both ear pinnas of mice. After 48 hours, the solution in which Evans' Blue solution and OA solution are blended equivalently was injected intravenously. After 30 min, the ear-pinnas were cut and the dye was extracted to quantify an dye leakage quantity by measuring an absorbance (λ=620 nm) by a spectrophotometer.

The results are shown in FIG. 3. The vertical axis of FIG. 3 shows the dye leakage quantity (μg), and the left is from the wild type mice and the right is from the PAR-2^{-/-} mice. As shown in FIG. 3, PCA reaction was induced both in the wild type and PAR-2^{-/-} mice, and no obvious effect of PAR-2 deletion was observed.

Contact dermatitis by delayed hypersensitivity has been known to be exerted by PC(2,4,6-trinitro-chlorobenzene), Ox(4-ethoxymethylene-2-phenyl-2-oxazoline-5-one) and other hapten antigens, and it has been known that inflammatory cells such as neutrophils, various cytokines and mediators are 3 involved in its reactions. Among them, tryptase which activates PAR-2 is one of serine proteases having a wide variety of physiological activities, and is reported to indirectly activate infiltration by neutrophils and eosinophils via PAR-2 expressed on the surfaces of vascular endothelial cells. Such events have been confirmed by the evidences that PAR-2 selectively activating peptides facilitate IL-8 release from keratinocytes and binding of NFκB to DNA and further induce rolling and adhesion of neutrophils on vascular endothelial cells. However, the role of PAR-2 delayed hypersensitivity still remain unclear.

In the present results, it is believed that absence of inflammatory response via PAR-2 contributes to remarkable suppression of contact dermatitis in the PAR-2^{-/-} mice.

It is also widely known that tryptase is released from mast cells in anaphylaxis and facilitates various inflammatory responses, but roles of PAR-2 in anaphylaxis are still unclear. In the present experiments using the typical cutaneous anaphylaxis model, the role of PAR-2 was examined, but no significant difference in PCA was observed between the wild type and PAR-2^{-/-} mice, indicating that involvement of PAR-2 in cutaneous anaphylaxis is insignificant.

From these results, it is shown that PAR-2 exhibits almost similar reactions as the wild type in PCA, and that PAR-2 is scarcely involved in the dermatitis models in which tryptase is released from mast cells. However, it has been shown that PAR-2 exhibited significantly different reactions from the wild type in the delayed hypersensitivity., and that deletion of PAR-2 exhibits evident suppressive actions. That is, the present invention provides a novel means to suppress delayed hypersensitivity by different mechanisms from suppression of tryptase release from mast cells, wherein delayed hypersensitivity can be suppressed by inhibiting PAR-2 activity or suppressing PAR-2 gene expression.

Therefore; the present invention first demonstrates that delayed hypersensitivity can be suppressed by substances which inhibit PAR-2 (antagonist) and can suppress expression of PAR-2 gene.

The invention also provides a novel method for measuring PAR-2 activity. PAR-2 belongs to G-protein-coupled receptor (GPCR) family, and produces inositol phosphates as a second messenger upon activation of the receptor. Based on this mechanism, the effects of subject substances in activation of PAR-2 can be determined quantitatively by quantifying produced inositol phosphates in PAR2-expressing cells by an ion chromatography or the like.

Activation of PAR-2 by trypsin or PAR-2 activating peptide, SLIGKV, could be determined by this method. Trypsin-mediated activation was inhibited by a trypsin inhibitor derived from soybeans, while SLIGKV-mediated activation was not affected. This manifests that inhibition of enzymes such as trypsin or tryptase is essentially different from the direct inhibition of the receptor.

The method for measuring PAR-2 activity of the invention can be widely applied, such as to screening of activation agents or inhibitors of PAR-2.

### BRIEF DESCRIPTION OF THE DRAWINGS

FIG. 1 shows the results of picryl chloride (PC)- or oxazolone (ox)-induced delayed allergic response experiments in wild type and PAR-2^{-/-} mice. FIG. 1A shows the results of PC-induced contact dermatitis model, and FIG. 1B shows the results of Ox-induced contact dermatitis model. The left and right sides denote the cases of the wild type mice and PAR-2^{-/-} mice, respectively in each Figure. The vertical axis in FIGS. 1A and 1B each denotes the difference of ear pinna thickness before and after the induction (×10⁻² mm). Each numerical value represents the mean±standard error. The symbol ** means that significant difference (P<0.01) exists.

FIG. 2 is color photographs instead of drawings, showing histological pathological features of specimens with hematoxylin-eosin staining of PC- or Ox-induced delayed allergic reactions in the wild type and PAR-2^{-/-} mice. The wild type mice (FIGS. 2A, 2C and 2E) and PAR-2 mice (FIGS. 2B, 2D and, 2F) were treated with solvent alone (olive oil; FIGS. 2A and 2B), PC (FIGS. 2C and 2D), or Ox (FIGS. 2E and 2F). The bar denotes 100 μm in length.

FIG. 3 shows the results of passive cutaneous anaphylaxis reaction tests in the wild type and PAR-2^{-/-} mice. The vertical axis in FIG. 3 denotes the leakage quantities of the dye (μg). The left and right are the cases of wild type and PAR-2^{-/-} mice, respectively. The columns and bars represent the mean±standard error in each group.

### DESCRIPTION OF THE PREFERRED EMBODIMENTS

The present invention provides a pharmaceutical composition for delayed hypersensitivity containing one or two or more active ingredients selected from the group consisting of inhibitors of PAR-2 and suppressive agents of PAR-2 gene expression, and a pharmaceutically acceptable carrier. The invention also provides a method for preventing/treating delayed hypersensitivity comprising administering a medicine containing an effective amount of one or two or more active ingredients selected from the group consisting of inhibitors of PAR-2 and suppressive agents of PAR-2 gene expression, to a patient with delayed hypersensitivity. Further, the invention provides use of one or two or more active ingredients selected from the group consisting of inhibitors of PAR-2 and suppressive agents of PAR-2 gene expression for producing a pharmaceutical composition for delayed hypersensitivity.

The inhibitor of PAR-2 or suppressive agent of PAR-2 gene expression of the invention is one which can inhibit PAR-2 activity or suppress expression of PAR-2 gene by a method for screening using PAR-2 expressing cells.

The inhibitor of PAR-2 or suppressive agent of PAR-2 gene expression of the invention has a suppressive action against delayed hypersensitivity, and is useful as a pharmaceutical composition for contact dermatitis, graft rejection, graft versus host disease, tuberculin reaction, granulation, tuberculosis, lepra, sarcoidosis, Crohn's disease, chronic ulcerative colitis, schistosomiasis, autoimmune diseases (such as rheumatoid arthritis, systemic lupus erythematosus, chronic ulcerative colitis, myasthenia gravis, insulin dependent diabetes mellitus, Hashimoto's thyroiditis, scleroderma, pernicious anemia), atopic dermatitis, asthma, chronic obstructive pulmonary disease, rhinitis, allergic conjunctivitis, food allergy, nephritis, and diseases with inflammatory infiltration by one or more types of neutrophils, macrophages, lymphocytes' and eosinophils.

The inhibitor of PAR-2 of the invention can be used alone or can be used in combination with the other pharmaceutically acceptable resolvents, excipients, binders, diluents and the like to be formulated into tablets, capsules, granules, powder, lotion, ointment, injection, suppository and the like. These preparations can be produced by the methods known in the art. For example, the preparations for oral administration can be produced by formulating in appropriate combination with resolvents such as gum tragacanth, gum arabic, sucrose ester, lecithin, olive oil, soybean oil, PEG400 and the like; excipients such as starch, mannitol, lactose and the like; binders such as sodium carboxymethyl cellulose, hydroxypropyl cellulose and the like; disintegrants such as crystal cellulose, calcium carboxymethyl cellulose and the like, lubricants such as talc, magnesium stearate and the like; fluidity improvers such as silicic anhydride light and the like.

The inhibitor of PAR-2 of the invention is administered orally or parenterally.

The dosage of the inhibitor of PAR-2 of the invention varies depending on body weight, age, sex, condition and the like of the patient, is usually from 0-01 to 1000 mg per day in the adult, and preferably it is preferred to administer from 0.1 to 100 mg by dividing into 1 to 3 times in a day.

The invention also provides a method for screening active ingredients for pharmaceutical composition for delayed hypersensitivity comprising screening subject substances for inhibitory action against PAR-2 or suppressive action against PAR-2 gene expression by contacting the subject substances to cells on which PAR-2 is expressed followed by determining expression or activity of PAR-2.

The cells expressing PAR-2 used for the screening method of the invention include, but not are limited to, for example, NCTC2544 cells, NEK293 cells, and the like, however, any cells which express PAR-2 of animals such as mouse, rat, human and the like can be used. For example, the screening can be performed by contacting the subject substance such as a candidate compound and the like with these cells expressing PAR-2 in a medium where the cells can be cultured and by measuring activity of PAR-2. The methods for measuring activity of PAR-2 is not specifically limited, but preferred is the measuring method by phosphorylation of inositol described below.

The invention provides a method for detecting or quantifying actions against PAR-2 activation by culturing PAR-2 expressing cells in the medium containing inositol followed by detecting or quantifying a quantity of inositol phosphates by the stimulation with PAR-2 in the medium to which a subject substance is added.

Inositol used for the method of the invention may be any of those which are phosphorylated by activation of PAR-2, but usually myo-inositol is preferable.

Also the method for measuring phosphorylation of inositol is not specifically limited, but the method of Oldham et al. reported to be simple and sensitive is preferable (Oldham, K. G., Polyphosphoinositide turnover. In Receptor-effector coupling—A practical approach, ed., Hulme, E. C., pp. 99-116, Oxford University Press, 1990). In this method, ³H-myo-inositol is used in the presence of lithium salt which is an inhibitor of inositol monophosphatase. The produced phosphorylated inositol is extracted with an organic solvent or TCA solution, and then is separated from free inositol using an ion exchanging chromatography.

In this way, the method employing inositol labeled with a radioisotope is relatively simple and preferred. The radioisotopes may be any of hydrogen, oxygen and carbon atoms of inositol, and for example, the hydrogen atoms can be labeled with radioactive ³H. PAR-2 activity can be measured by detection or quantification phosphorylated inositol using such labeled inositol. This method of the invention can screen whether the subject substance has an inhibitory action against PAR-2 or a suppressive action against PAR-2 gene expression or not.

### EXAMPLES

The invention is more specifically described by the following examples, but the technical scope of the invention is not limited to these examples.

The mice used in the following experiments were PAR-2^{-/-} and its wild type male mice derived from a hybrid strain of C57BL/6 and 129/O1a bred in our laboratory, of ages from 6 to 9 weeks, were bred at 23±3° C., and were given feeds and water ad libitum.

### Example 1

### Examination on Skin Atrophy

The thickness of ear pinnas was measured in non-treated wild type and PAR-2^{-/-} mice. The average values of each 10 mice are:

Wild type mice 22.7±0.4 (×10⁻² mm; the mean±standard error)

PAR-2^{-/-} mice 22.0±0.4 (×10⁻² mm; the mean±standard error)

and no statistical difference was observed between the two groups. Also no difference was macroscopcally observed, indicating that inhibition of PAR-2 did not cause skin atrophy.

### Example 2

### Picryl chloride (PC) (2,4,6-trinitro-chlorobenzene)-induced Contact Dermatitis Model

After shaving hairs, each 10 mice of PAR-2^{-/-} and wild type groups were sensitized by applying 100 μl of 7% PC-ethanol solution on the abdominal part. After 6 days, the thickness of both ear pinnas was measured using a dial thickness gauge (Peacock G-1A, OZ-AKI MFG. Co. Ltd.). Then, induction was performed by applying 20 μl of 1% PC-olive oil or solvent alone on the both sides of the ear pinnas, and then the thickness of ear pinnas was measured after 6, 24, and 48 hours. The difference of ear pinna thickness before and after the induction was calculated to be an indicator of edema. The results are shown in Table 1 described below.

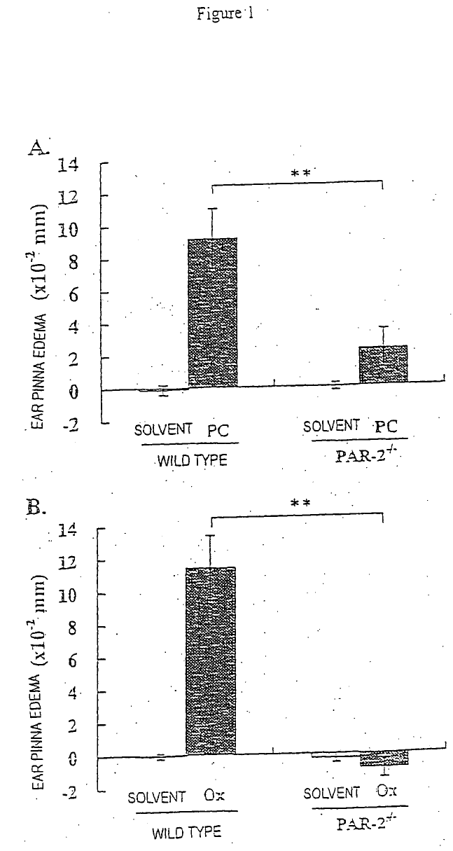

The result after 24 hours is represented as a graph in FIG. 1A. By applying PC on the ear pinnas of the wild type mice, incidences of edema were observed, of which peak was at 24 hours after the induction. Edema and rubefaction of the ear pinnas were also observed with the naked eye. These incidences of edema were obviously inhibited in the PAR-2^{-/-} mice (FIG. 1A). No effect of application with solvent (olive oil) was found.

### Example 3

### Oxazolone (Ox)

### (4-ethoxymethylene-2-phenyl-2-oxazoline-5-one)-induced Contact Dermatitis Model

After removing hairs, each 5 mice of PAR-2^{-/-} and wild type groups were sensitized by applying with 100 μl of 0.5% Ox-ethanol solution on the abdominal part. After 5 days, the thickness of the ear pinnas before the induction was measured using the dial thickness gauge. Then, the induction was performed by applying with 20 μl of 0.5% Ox-acetone solution or solvent alone, and the thickness of the ear pinnas was measured after 6, 24, and 48 hours. The difference of ear pinna thickness before and after the induction was calculated to be an indicator of edema. The results are shown in Table 1 in conjunction with the above results.

TABLE 1

| | | Hours after the challenge | | |
|---|---|---|---|---|
| Genotype | n | 6 | 24 h | 48 h |
| PC-induced contact dermatitis | | | | |
| Wild type | 10 | 1.1 ± 0.3 | 9.2 ± 1.8 | 7.3 ± 1.3 |
| PAR-2^{-/-} | 10 | 0.4 ± 0.3 | 2.3 ± 1.2** | 3.5 ± 1.3 |
| Ox-induced contact dermatitis | | | | |
| Wild type | 5 | 1.0 ± 0.6 | 11.4 ± 2.0 | 9.3 ± 0.7 |
| PAR-2^{-/-} | 5 | −0.1 ± 0.4 | −0.9 ± 0.6** | 4.1 ± 1.8* |

The numerical values denote the mean ± standard error of the ear pinna edema. (×10⁻² mm)
Each numerical value in Table 1 represents the mean value standard error of the ear pinna edema. The symbols *(P < 0.05) and **(P < 0.01) denote the significant difference.

The result after 24 hours is shown in FIG. 1B as a graph. As shown in FIG. 1B and as is the case with PC, incidences of edema were observed by applying Ox on the ear pinnas of the wild type mice. The edema reaction was completely inhibited in the PAR-2^{-/-} mice. No effect of application of solvent (acetone) was found.

### Example 4

### Histological Examination

The ear pinnas were exsected from anesthetized mice used in the examinations for PC- and Ox-induced contact dermatitis models, fixed with neutral buffer formalin and embedded with paraffin to make thin sections, which were stained with hematoxylin-eosin. The effects of PAR gene deletion were histologically observed on PC- or Ox-induced contact dermatitis.

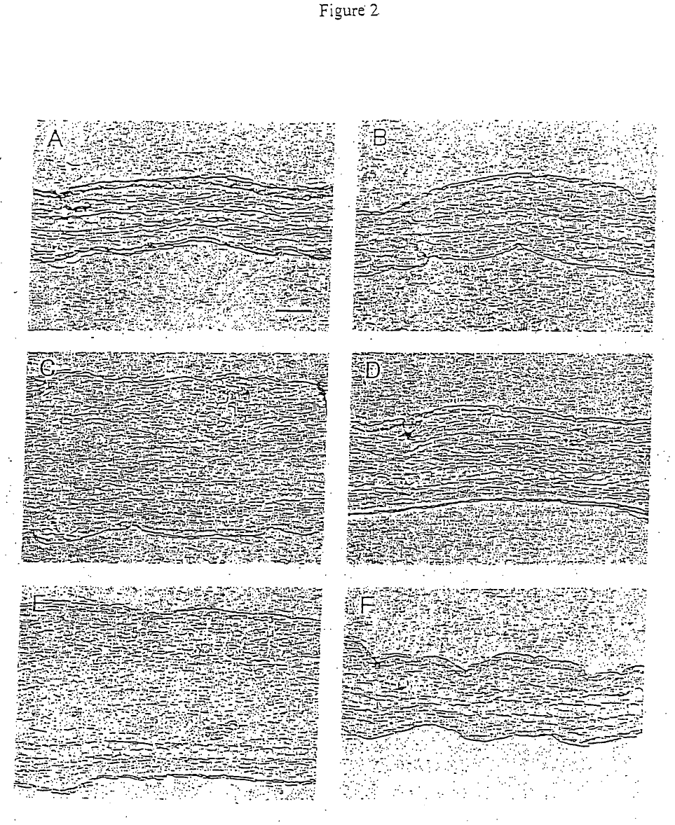

The histological and pathological features of 24 hours after the challenge with PC or Ox in the wild type and PAR-2^{-/-} mice are shown in FIG. 2 as color photographs instead of the drawings. Edema and infiltration by inflammatory cells such as neutrophils, macrophages lymphocytes, eosinophils and the like were observed by applying PC or Ox on the ear pinnas of the wild type mice (FIGS. 2C and 2E). Such edema and infiltration by inflammatory cells such as neutrophils, macrophages, lymphocytes, eosinophils and the like were remarkably suppressed in the PAR-2^{-/-} mice (FIGS. 2D and 2F).

### Example 5

### Passive Cutaneous Anaphylaxis (PCA) Model

After anesthetizing mice with ether, anti-OA anti serum (derived from BALB/c mice, PCA Titer 16 folds) diluted with saline was subcutaneously injected at 5 μl/site in both ear pinnas using a microsyringe. The solution in which 1%-Evans blue solution and 0.2% OA solution were blended equivalently was intravenously administered at a volume of 10 ml/kg after 48 hours. The ear pinna was cut off after 30 min, to which 0.25 ml of 1N KOH was added and incubated at 37° C. overnight. Then, 0-375 mL of 6N phosphoric acid and 1.125 mL of acetone were further added, and centrifuged (at 3,000 rpm for 10 min) to extract the dye. The leakage quantity of the dye was quantified by determining absorbance (λ=620 nm) using a spectrometer (UV-2200, Shimadzu Corporation).

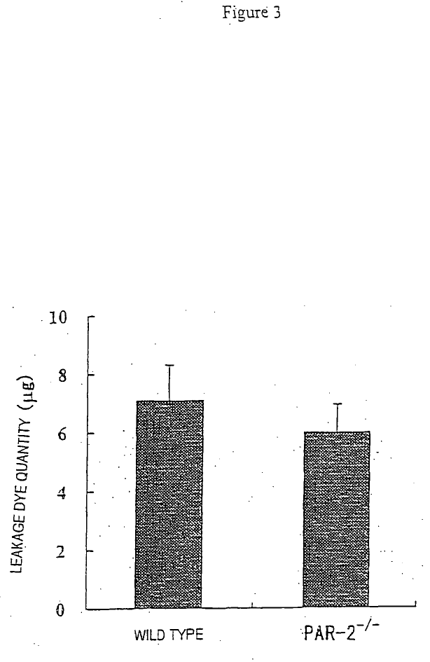

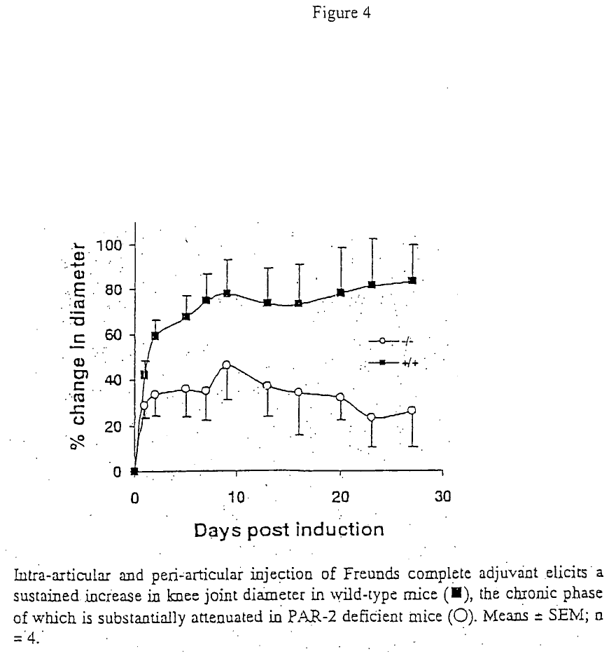

The results are shown in FIG. 3 as a graph. The PCA reaction was exerted in the wild type and PAR-2^{-/-} mice, and the levels of their reactivity were compared As shown in FIG. 3, PCA reaction was exerted both in the wild type and PAR-2^{-/-} mice, and a clear effect of PAR-2 gene deletion was not found.

### Example 6

### The Assay Method of PAR-2 Using Inositol Phosphates as an Indicator

NCTC2544 cells with stably high expression of PAR-2 were cultured in the serum-free medium containing 2 mCi/ml of [³H]-myo-inositol for 18 hours. Lithium chloride (final 5 ml) was added 30 min before the stimulation, and subsequently cells were stimulated with trypsin (1 to 30 nM) or the PAR-2 agonist peptide, SLIGKV (10 to 300 μM). After 45 min the lipid component was extracted with methanol and separated using an anion exchanging resin (AG1-X, formate form). Then the yield of inositol phosphates was determined in a scintillation counter. The subject substance was added 15 min before the stimulation.

From the results of the above studies, it is shown that the inhibitor of PAR-2 and the suppressive agent of PAR-2 gene expression are useful as the suppressive agent for delayed hypersensitivity without any side effect. The present invention provides the novel pharmaceutical composition with few side effects for delayed hypersensitivity by suppressing causative action of delayed hypersensitivity which is elucidated by the invention. In particular, it is useful as the pharmaceutical composition for contact dermatitis, graft rejection, graft versus host disease, tuberculin reaction, granulation, tuberculosis, lepra, sarcoidosis, Crohn's disease, chronic ulcerative colitis, schistosomiasis, autoimmune diseases (such as rheumatoid arthritis, subchronic rheumatoid arthritis, juvenile subchronic rheumatoid arthritis, systemic lupus erythematosus, chronic ulcerative colitis, myasthenia gravis, insulin dependent diabetes mellitus, Hashimoto's thyroiditis, scleroderma, pernicious anemia), psoriatic arthritis, atopic dermatitis, asthma, chronic obstructive pulmonary disease, rhinitis, allergic conjunctivitis, food-allergy, nephritis and diseases with inflammatory cell infiltration by one or more types of neutrophils, macrophages, lymphocytes and eosinophils, all of which are particularly caused by the delayed hypersensitivity reaction. Further, it does not cause skin atrophy which is one of side effects of topical steroid drugs currently and widely used for clinical treatment, resulting in a lower potential for manifestation of side effects.

The invention also provides the screening method for the pharmaceutical composition of delayed hypersensitivity using cells expressing PAR-2, by demonstrating, that PAR-2 is involved in delayed hypersensitivity reaction. The screening method of the invention enables to simply find the novel pharmaceutical composition of delayed hypersensitivity with few side effects. Additionally, the invention provides the measuring method of PAR-2 activity. The measuring method of the invention can determine PAR-2 activity simply and accurately, and enables to detect or quantify the action of the subject substance For PAR-2.

### Example 7

### Chronic Joint Inflammation is Attenuated in PAR-2 Deficient Mice

Intra- and peri-articular administration of Freunds complete adjuvant (FCA) resulted in a substantial increase in knee joint diameter in wild type mice which was sustained over a four week period. However, this chronic inflammatory response was markedly inhibited in PAR-2 −/−mice (FIG. 4), differing significantly from wild type mice (P<0.001; 2-way ANOVA). All animals showed an initial inflammatory response 24 hrs after injection of FCA, but the chronic inflammatory phase evident in the WT animals was notably absent in PAR-2/-mice.

1. A pharmaceutical composition for delayed hypersensitivity containing one or two or more active ingredients selected from the group consisting of inhibitors of PAR-2 and suppressive agents of PAR-2 gene expression and a pharmaceutically acceptable carrier.

2. The pharmaceutical composition for delayed hypersensitivity according to claim 1, wherein the pharmaceutical composition for delayed hypersensitivity is a suppressive agent for contact skin hypersensitive reaction and/or allogenic graft rejection.

3. The pharmaceutical composition for delayed hypersensitivity according to claim 1, wherein the pharmaceutical composition for delayed hypersensitivity is a pharmaceutical composition for contact dermatitis, graft rejection, graft versus host disease, tuberculin reaction, granulation, tuberculosis, lepra, sarcoidosis, Crohn's disease, chronic ulcerative colitis, schistosomiasis, autoimmune diseases (such as rheumatoid arthritis, subchronic rheumatoid arthritis, juvenile subchronic rheumatoid arthritis, systemic lupus erythematosus, chromic ulcerative colitis, myasthenia gravis, insulin dependent diabetes mellitus, Hashimoto's thyroiditis, scleroderma, pernicious anemia), psoriatic arthritis, atopic dermatitis, asthma, chronic obstructive pulmonary disease, rhinitis, allergic conjunctivitis, food allergy, nephritis, and diseases with inflammatory infiltration by one or more types of neutrophils, macrophages, lymphocytes and eosinophils.

4. One or two or more active ingredients selected from the group consisting of inhibitors of PAR-2 and suppressive agents of PAR-2 gene expression for use in a method of preventing or treating delayed hypersensitivity.

5-7. (canceled)

8. Method of preventing or treating delayed hypersensitivity comprising administering to an individual in need thereof a non-toxic effective amount of one or two or more active ingredients selected from the group consisting of inhibitors of PAR-2 and suppressive agents of PAR-2 gene expression.

9. Method of preventing or treating a condition as defined in claim 2 comprising administering to an individual in need thereof a non-toxic effective amount of one or two or more active ingredients selected from the group consisting of inhibitors of PAR-2 and suppressive agents of PAR-2 gene expression.

10. A screening method comprising screening a subject substance for an inhibitory action against PAR-2 or a suppressive action against PAR-2 gene expression by contacting the subject substance with cells expressing PAR-2 and by determining expression or activity of PAR-2.

11. The method according to claim 10, wherein the type of cells expressing PAR-2 is NCTC2544 cell.

12. The method according to claim 10, wherein the method for screening for an inhibitory action against PAR-2 or a suppressive action against PAR-2 gene expression is achieved by a PAR-2 assay method utilizing production of inositol phosphates as an indicator.

13. A detecting or quantifying method of PAR-2 activation by detecting or quantifying a quantity of inositol phosphates by PAR-2 expressing cells in culture to which the subject substance is added following to a culture of the PAR-2 expressing cells in a medium containing inositol.

14. The method according to claim 13, wherein a type of cells expressing PAR-2 is NCTC2544 cell.

15. A substance which has inhibitory action against PAR-2 or a suppressive action against PAR-2 gene expression identified by the method of.

16. Method according to claim 10 which further comprises formulating the substance thus identified which has inhibitory action against PAR-2 or a suppressive action against PAR-2 gene expression into a composition for preventing or treating.

17. (canceled)

18. Method according to claim 10 which further comprises administering a non-toxic effective amount of the identified substance to an individual in need thereof for preventing or treating delayed hypersensitivity.

19. (canceled)

20. A method of treating a patient suffering from or susceptible to a disease or disorder that is contact dermatitis, graft rejection, graft versus host disease, tuberculin reaction, granulation, tuberculosis, lepra, sarcoidosis, Crohn's disease, chronic ulcerative colitis, schistosomiasis, an autoimmune disease, psoriatic arthritis, atopic dermatitis, asthma, chronic obstructive pulmonary disease, rhinitis, allergic conjunctivitis, food allergy, nephritis, or a disease having inflammatory infiltration by one or more types of neutrophils, macrophages, lymphocytes and eosinophils, comprising:
administering to the patient an effective amount one or more inhibitors of PAR-2 and/or one or more suppressive agents of PAR-2 gene expression.

21. The method of claim 20 wherein the patient is suffering from rheumatoid arthritis, subchronic rheumatoid arthritis, juvenile subchronic rheumatoid arthritis, systemic lupus erythematosus, chromic ulcerative colitis, myasthenia gravis, insulin dependent diabetes mellitus, Hashimoto's thyroiditis, scleroderma, or pernicious anemia.

22. A method of treating a patient suffering from or susceptible to delayed hypersensitivity, comprising:
administering to the patient an effective amount one or more inhibitors of PAR-2 and/or one or more suppressive agents of PAR-2 gene expression.

23. A method of treating a patient suffering from or susceptible to contact skin hypersensitive reaction and/or allogenic graft rejection, comprising:
administering to the patient an effective amount one or more inhibitors of PAR-2 and/or one or more suppressive agents of PAR-2 gene expression.
